(19) United States
(12) Patent Application Publication
(10) Pub. No.: US 2021/0137506 A1
(43) Pub. Date: May 13, 2021

(54) METHODS AND SYSTEMS FOR COLLECTING CELLS AND BIOLOGICAL SAMPLES FOR SCREENING

(71) Applicant: Smart Pap, Inc., Menlo Park, CA (US)

(72) Inventor: Avnesh Thakor, Menlo Park, CA (US)

(73) Assignee: Smart Pap, Inc., Menlo Park, CA (US)

(21) Appl. No.: 17/156,386

(22) Filed: Jan. 22, 2021

Related U.S. Application Data
(63) Continuation of application No. PCT/US2019/043440, filed on Jul. 25, 2019.
(60) Provisional application No. 62/711,524, filed on Jul. 28, 2018.

(51) Int. Cl. A61B 10/02; A61B 10/00

(52) U.S. Cl. CPC A61B 10/0291; A61B 2010/0216; A61B 2010/0003; A61B 10/0096

(57) ABSTRACT

A device for self-collection of a cervical sample includes an enclosure, a sample-collecting head, and a slider and a knob on the enclosure. The enclosure can be gripped by a user in one hand and inserted into the user's vaginal canal. The enclosure has an opening at one end, and a sample-collecting head can be advanced and retracted through the opening. The slider is on a side of the enclosure and is coupled to advance and retract the sample-collecting head, typically using a thumb. The knob is rotated by the user to rotate the sample-collecting head while the enclosure remains in the user's vaginal canal.

### CROSS-REFERENCE TO RELATED APPLICATION

This application is a continuation of PCT Application No. PCT/US2019/43440 (Attorney Docket No. 54815-703.601), filed Jul. 25, 2019, which claims the benefit of U.S. Provisional Patent Application No. 62/711,524 (Attorney Docket No. 54815-703.102), filed on Jul. 28, 2018, the full disclosures of which are incorporated herein by reference.

### BACKGROUND OF THE INVENTION

1. Field of the Invention. The present invention relates generally to methods and systems for collecting cells and biological samples from a female patient's vaginal canal and/or a cervix and more particularly to methods and systems which allow the patient to self-collect the sample, preserve the sample, and send the collected samples to a laboratory or other remote location for analysis.

Cervical cancer screening relies on detecting dysplastic or metaplastic squamous or adenocarcinoma cells in a cell sample obtained from the patient's cervical os. The cervical cells are obtained in a “pap smear” performed by a trained medical professional. Pap smears are generally performed annually and requires that the patient make an appointment at a medical office, take time away from work or family to go to the appointment, endure the embarrassment and physical discomfort of having one or more medical professionals view and access her vagina with a speculum. In some cases, the patient may experience bleeding after the procedure, as a result of trauma induced by the tools in use today.

It is therefore an object of the present invention to provide methods and systems which allow an individual female patient to self-collect biological samples, such as vaginal and cervical samples, by herself without assistance from others. The methods and systems should be useful both in clinical environments where the self-collected sample could be given directly by the patient to a testing laboratory and in non-clinical environments, such as the patient's home, where the sample could be preserved and transported for testing at a remote laboratory. In particular, the methods and systems should allow an individual patient to conveniently remove the sample from the collector, place the sample in a container with a preservative/culture solution, and send the container to the laboratory while avoiding contamination of the sample that could adversely affect test results. The methods and systems should be less invasive and should reduce patient embarrassment and discomfort. The methods and systems for sample collection should be suitable for collecting cells for use in pap smears as well as being compatible with screening for a variety of other diseases and conditions. At least some of these objectives will be met by the inventions described hereinbelow.

2. Listing of the Background Art. Relevant patents and publications include US2017042518; US2013338533; US2012157878; US2002068881; U.S. Pat. Nos. 8,801,628; 8,672,861; 8,460,209; 8,152,739; 5,445,164; 5,191,899; 4,700,703; WO2016/160454; WO2007/045896; and CN201023099. A cervical cell collector including a brush with a plurality of cell-collecting bristles is commercially available under the tradename Wallach's Papette® Cervical Cell Collector (coopersurgical.com).

### SUMMARY OF THE INVENTION

The present invention provides methods, devices and systems which allow an individual female patient to self-collect biological samples, such as vaginal and cervical samples, by herself without assistance from others. The methods and systems of the present invention are useful in clinical environments where the self-collected sample could be given directly by the patient to a testing laboratory, in office environments where the patient can give the sample to a nurse or physician to follow a standard of care workflow, and in non-clinical environments, such as the patient's home, where the sample could be preserved and transported for testing at a remote laboratory. Using the methods, devices and systems of the present invention allows an individual patient to conveniently remove the sample from the sample collector, place the sample in a container with a preservative/culture, and send the container to the laboratory while avoiding contamination of the sample that could adversely affect test results. The methods and systems are less invasive and reduce patient embarrassment and discomfort. The methods and systems for sample collection should be suitable for collecting cells for use in pap smears as well as being compatible with screening for a variety of other diseases and conditions.

In a first aspect, the present invention provides a device for self-collection of a sample, such as a cervical sample, a vaginal sample, or other tissue or cellular sample that can be collected from the vaginal canal near the cervix. The device comprises an enclosure, a sample-collecting head, a translational actuator, and a rotational actuator. The enclosure is configured to be gripped in a hand of a user and to be inserted into the user's vaginal canal. The sample-collecting head is configured to be advanced and retracted through an opening at a distal head of the enclosure, and the translational actuator is disposed on a side of the enclosure. The translational actuator is coupled to the sample-collecting head and is configured to be engaged by one or other of the user's hands, usually by the thumb of the hand holding the enclosure, to advance the sample-collecting head through the opening to a location proximate the user's cervical os and retract the sample-collecting head through the opening into an interior of the enclosure. In some embodiments, the translational actuator can be a slider or other axially translatable feature. In other embodiments, the translational actuator can be a wheel which can be manually rotated to axially advance a shaft which carries the sample-collecting head. The rotational actuator is configured to be rotated by one or other of the user's hands, usually the hand not holding the enclosure, to rotate the sample-collecting head while the enclosure is in the user's vaginal canal and being held by one or other of the user's hands.

Optionally, indicators showing the extent of axial advancement of the sample-collecting head may be provided on the enclosure. For example, a window with a sliding bar or ribbon may be provided to inform the user of to axial position of the sample-collecting head. Such indicators will be particularly useful with wheel-type translational actuators that provide no inherent positional information.

Still other axial translation mechanism may comprise spiral stems that can be driven by an eccentric disc, flexible stems that can be furled and unfurled on a roller or wheel, and ratcheted stems that can be driven by an engagement wheel.

Further optionally, mechanisms may be provided to vibrate the sample-collecting head, either as an adjunct to rotation or in some instances an alternative to rotation. For example, a piezoelectric transducer could be coupled to a stem which carries the sample-collecting head in order to impart a rotational, axial, or other vibrational motion component to the sample-collecting head.

In particular embodiments of the present invention, a distal region of the enclosure tapers to a distal tip, typically being shaped or configured to conform to a vaginal anatomy. The opening in the enclosure is typically disposed at the tapered distal tip. In other embodiments, the enclosure may be straight, non-tapered, or have other shapes compatible with the anatomy.

In other embodiments of the present invention, the sample-collecting head may comprise any of the variety of shapes, materials, collection features, surface treatments, and the like. Suitable shapes include disks, spears, triangles, pyramids, and the sample-collecting heads may comprise brushes, sponges, protrusions, fingers, bristles, prongs, protrusions, fingers, lipped edges, fanned prongs or fingers, spirals, reticulated polyurethane meshes, and the like. Suitable materials of construction for the sample-collecting head include polyurethanes, cellulose and cellulosic materials, gel foams, gelatin, polytetrafluoroethylenes (PTFE's), graphenes, silicones, polydimethylsiloxane, collagen-based materials (e.g. cryogels), and the like. The sample-collection surfaces of the sample-collecting head may be coated, treated, or otherwise modified to provide sufficient abrasiveness to collect and retain cells without damaging tissue. Other surface treatments and materials may be incorporated to promote sterility, enhance elasticity, allow folding, and the like. The material(s) of construction should also be suitable for storage in a preservative and/or cell culture solution of type used to store the samples when the sample-collecting head is being sent to a central laboratory for analysis, as described hereinbelow. Sponges, if used, should be formed from a material that will not degrade and/or disintegrate or have impurities that could be released to degrade the sample.

In specific embodiments, as described in more detail below, the sample-collecting heads of the present invention may comprise a foldable backing having a forwardly or distally facing sample collection surface. The sample collection surface may comprise any of the surface features such as brushes, sponges, protrusions, fingers, bristles, prongs, protrusions, fingers, lipped edges, fanned prongs or fingers, spirals, or the like, as described previously. The foldable backing allows the head to be folded when present in the interior of the enclosure so that the sample-collecting head may unfold and deploy as it advanced through the opening into the vaginal canal. The sample-collecting head will typically be further configured to refold when retracted back into the interior of the enclosure, where such refolding protects the sample collection surface and any features thereon from mechanical disruption in order to assure preservation of the sample as the sample-collection head is retracted. The foldable or other backing or surface of the sample-collecting head will typically be mounted on a shaft, stem, or other coupling arm which is rotated and axially advanced relative to the enclosure. Usually a string or filament or other tether will also be attached to the sample-collecting heads to allow retrieval of the sample-collecting head should it ever detach from the coupling arm while in the user's vaginal canal. The tether will typically be brought out to the enclosure so that it is accessible in the case of sample-collecting head detachment.

In further specific instances, the sample collection devices of the present invention may comprise a slidable carriage translatably disposed within the housing. The slidable carriage is coupled to both the sample-collecting head and the translational actuator so that advancing and retracting the translational actuator advances and retracts the sample-collecting head. In particular, the translational actuator may comprise a slider having a thumb button and a coupling arm which attaches the thumb button to the slidable carriage. The coupling arm is slidably received in an axial slot in a wall of the housing, allowing the user to advance and retract the thumb button using the thumb of the hand which is holding the enclosure. In further specific instances, the axial slot may have ratchets formed along an axial edge thereof. The coupling arm may be biased to close against or otherwise engage the ratchets to immobilize the slider and the slidable carriage when the thumb button is released and to disengage the ratchets when the thumb button is depressed to advance and retract the slider and the slidable carriage.

In still further instances, the rotational actuator of the devices of the present invention may be coupled to the sample-collecting head by a rotatable barrel and a shaft where the shaft is keyed to the rotatable barrel to allow axial extension and retraction of the sample-collecting head relative to the barrel while the barrel is being rotated by the rotational actuator. Preferably, the rotational actuator is coupled to the rotatable barrel by a gear train which rotates the barrel from two to 12 times, typically from four to 10 times, often about five times, for each rotation of the rotational actuator. Such rotational amplification helps assure that the user will rotate the sample-collecting head a sufficient number of times in order to obtain a useful sample for analysis.

Optionally, features may be added to the sample collectors of the present invention to provide user feedback regarding the number of sample-collecting head rotations and/or to provide limits on the number of rotations of the sample-collecting head in a single use. For example, a rotation counter could be provided, such as a “clicker” that provides an audible sound at each rotation or a numeric counter that increments at each rotation. Alternatively or additionally, a mechanical limit could be provided which prevents further rotation of the sample-collecting head beyond an upper limit, such as 12 rotations. Over-rotation of the sample-collecting head could damage tissue surrounding the cervix.

In other embodiments, the axial actuator and rotational actuator may both comprise wheels or rings. A first wheel or ring could engage the coupling arm to axially translate while a second wheel or ring could be coupled to the coupling arm to directly or indirectly provide rotation.

In still other embodiments, the coupling arm could be driven by battery-powered or other motors to achieve axial and rotational actuation of the sample-collecting head. A variety of gear linkages could be employed, and operation of the motors could be manual, programmed, or a combination thereof.

In other specific instances, the enclosures will have an insertion stop on an exterior of the enclosure, typically a flange circumscribing the body of the enclosure, at a location spaced proximally of the distal end. The insertion stop allows the user to insert the sample collector by an amount which places the distal tip a proper distance from the cervical os when the enclosure when the insertion stop engages tissue surrounding the vaginal opening. In certain instances, the insertion stop may be positionable at different locations along the length of the enclosure and/or sample collectors having different positioning of the insertion stops may be provided to allow a user to select a self-collection device compatible with the user's anatomy. In some cases, physicians or other prescribing individuals may help the user select a sample collector having a proper insertion depth defined by the insertion stop.

In a second aspect, the present invention provides systems for collecting and transporting a cervical sample. The systems may comprise any of the self-collection devices described previously in combination with a collection receptacle having an aperture configured to receive a distal end of the enclosure. The aperture is configured to receive the distal end of the enclosure to allow the sample-collecting head to be advanced through the opening of the enclosure and into an interior volume of the collection receptacle. The systems further include a cover configured to be sealed over the aperture of the collection receptacle after the sample-collecting head has been detached from the device and released into the interior volume of the collection receptacle.

In specific instances of the collecting and transporting systems of the present invention, the aperture will be configured to seal about an exterior of the enclosure of the device for self-collection to prevent or inhibit contamination of the sample as it is being released into the collection receptacle. For example, the aperture may be sized so that the tapered distal region of the device for self-collection frictionally engages and forms a seal between the aperture and the exterior of the enclosure.

In further instances, the collection receptacles of the systems for collecting and transporting cervical samples of the present invention may comprise or otherwise integrate cutters configured to detach the sample-collecting head from the device for self-collection of a cervical sample while the aperture remains sealed about the exterior of the enclosure, further preventing or inhibiting contamination of the sample-collection head before sealing the collection receptacle with the cover. In particular, using a sterile collection receptacle having both the cover and the integrated cutters allows the sample to be collected and released into the receptacle without any need for the user to touch the sample-collecting head with her hands or potentially contaminating tools.

Alternative sample-collecting head detachment mechanisms include interconnected hooks, electrolytically degradable links, and the like. For example, a release button could be provided on an exterior the device enclosure to deliver a charge to an electrolytically degradable link in the stem or elsewhere to detach the sample-collecting head after it is immersed in the preservative fluid.

In a third aspect, the present invention provides methods for self-collection of the cervical sample by a user. Such methods comprise a series of steps performed by the user, typically using either or both hands. The user first grips the enclosure, typically using a single hand, and inserts a distal end of the enclosure into her vaginal canal. A sample-collecting head is then advanced from an interior of the enclosure through an opening to engage tissue proximate the user's cervical os. The sample-collecting head is then rotated while the enclosure remains in the user's vaginal canal to collect sample from the tissue. After the sample is collected, the sample-collecting head is retracted back into the interior of the receptacle and the distal end of the enclosure is withdrawn from the vaginal canal. All such steps may be performed by the user without help or attendance by others.

In specific aspects of the methods of the present invention for self-collection of the cervical sample, the user may grip the enclosure in one hand and insert the distal end of the enclosure into the vaginal canal using that same hand. The user may then use either hand, but typically using the hand which holds the enclosure, to advance a translational actuator on an exterior of the enclosure to advance the sample-collecting head from an interior of the enclosure through the opening to engage tissue proximate the user's cervical os. After engaging the sample-collecting head against the tissue, the user may use either hand, but typically using the hand which doesn't hold the enclosure, to rotate a rotational actuator on an exterior of the enclosure to rotate the sample-collecting head to collect a sample from the tissue proximate the user's cervical os. In particular instances, the gear train may cause the sample-collecting head to rotate from 2 to 12 times for each rotation of the rotational actuator.

After the sample has been collected. The user may use the one hand to retract the translational actuator on the exterior of the enclosure to retract the sample-collecting head back through the opening into the interior of the enclosure. The user may then withdraw the enclosure, typically using the one hand, from the vaginal canal after the sample-collecting head has been retracted.

In preferred instances, the sample-collecting head will be foldable so that it may be folded with a sample-collection surface protected while it is within the interior of the enclosure. In particular, as the sample-collecting head is drawn back into the interior of the enclosure through the opening, the foldable backing will engage the periphery of the opening to protect the sample-collecting surface from frictional engagement which could result in loss of sample.

After the sample has been collected, and the enclosure removed from the patient's vaginal canal, a distal end of the enclosure may be inserted through an aperture of a collection receptacle which contains a cell preservative and/or microbial culture solution, depending on the nature of the test. Typically, the distal end of the enclosure will be sealed against the aperture on the receptacle as the sample-collecting head is advanced through the opening on the enclosure and into the preservative/culture solution. Sealing of the enclosure against the aperture of the collection receptacle reduces the risk of contamination.

The methods of the present invention typically further comprise detaching the sample-collecting head from the enclosure after the enclosure is sealed to reduce the risk of contamination. Detaching the sample-collecting head typically comprises actuating one or more cutting blades disposed in the interior of the enclosure. Preferably, the cutting blades may be actuated using pushers or other components present on the exterior of the collection receptacle to further reduce the risk of contamination. Usually the blades will be stowed in the collection receptacle or otherwise rendered safe after use.

In alternate embodiments, detaching the sample-collecting head from the enclosure after the enclosure is sealed may comprise electrolytically degrading a link, or disengaging a mechanical link, securing the sample-collecting head to the enclosure. Specific examples are provided hereinafter.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B are perspective views of the sample collector of the present invention shown with a sample-collecting head in its retracted configuration and its extended configuration, respectively.

FIGS. 2A and 2B are cross-sectional views of the sample collectors of FIGS. 1A and 1B, respectively.

FIG. 3 is an exploded view of the sample collector of FIGS. 1A and 2A, illustrating the internal components.

FIG. 4 is a perspective, cross-sectional view of the proximal end of the sample collector of FIGS. 1A and 2A.

FIG. 5 is a detailed, cross-sectional view of the proximal end of the sample collector of FIGS. 1A and 2A.

FIGS. 6A and 6B illustrate a first embodiment of a sample-collecting head of the present invention shown in unfolded and folded configurations, respectively.

FIGS. 7A and 7B illustrate a second embodiment of a sample-collecting head of the present invention shown in unfolded and folded configurations, respectively.

FIGS. 8A and 8B illustrate a third embodiment of a sample-collecting head of the present invention shown in unfolded and folded configurations, respectively.

FIGS. 9A and 9B illustrate a fourth embodiment of a sample-collecting head of the present invention shown in unfolded and folded configurations, respectively.

FIGS. 10A and 10B illustrate a fifth embodiment of a sample-collecting head of the present invention shown in unfolded and folded configurations, respectively.

FIGS. 11A and 11B illustrate a sixth embodiment of a sample-collecting head of the present invention shown in unfolded and folded configurations, respectively.

FIGS. 12A through 12F illustrate exemplary steps of the present invention using the sample-collecting device of the previous figures for collecting a cell sample by a user.

FIGS. 13A through 13F illustrate the steps for transferring a sample-collection head from a sample collector into a collection receptacle in accordance with the principle of the present invention.

FIG. 14 is a perspective view of a sample collector of the present invention shown with an alternative sample-collecting head actuation configuration.

FIGS. 15A and 15B are partial side views of the sample collector of FIG. 14 shown with the sample-collecting head actuator shown in its retracted configuration and its extended configuration, respectively.

### DETAILED DESCRIPTION OF THE INVENTION

Referring now to FIGS. 1A and 1B, a sample collector 10 constructed in accordance with the principles of the present invention comprises a hand-held enclosure 12 having a distal end 14 and a proximal end 16. A distal region 18 of the sample collector 10 is typically tapered, curved, and configured to anatomically conform to a user's vaginal canal in a manner that allows the user to self-collect cell samples, typically from at or near the user's cervical os, as will be described in detail herein below.

The enclosure 12 typically includes a flange or other insertion stop 22 on its exterior surface. The flange 22 will be located at a pre-selected distance proximal of the distal end 14 of the enclosure, where the distance is selected to provide a controlled insertion length for the distal region 18, typically from 1 cm to 10 cm, usually from 2 cm to 8 cm.

The sample collector 10 further includes an axial actuator, typically a slider 24, which includes a thumb button 26 which slides in an axial slot 28. A rotational actuator, such as rotatable knob 30, is typically disposed at the proximal end of the enclosure 12. As best seen in FIG. 1B, the slider 24 or other axial actuator or other axial actuator can be advanced in the distal direction to deploy a sample-collecting head 34 through a distal opening 32 in the distal end 14 of the enclosure 12. The sample-collecting head 34 is typically attached or otherwise disposed at a distal end of a coupling arm 36 which is coupled to the slider 24 as described in more detail below.

Referring now to FIGS. 2A, 2B, and 3, exemplary internal mechanisms for axially advancing and retracting the sample-collecting head 34 and coupling arm 36 will be described. As can be seen in FIG. 3, the enclosure 12 may be formed as a pair of “half shells” 40 which may be held together using internal posts, screws, welding, adhesives, and the like, in a conventional manner. The details of the attachment are not shown in the drawings.

A carriage 42 having a base 44 at a distal end thereof is attached to the thumb button 26 by a lever arm 50. The lever arm is resiliently or pivotally attached to the base 44 so that the button 26 may be depressed by pressing downwardly, typically by a user's thumb, so that the button will spring back when the downward pressure is removed. A cam element 52 is carried on a lower surface of the button 26, and a pair of channel guides 48 are provided which allow the carriage 42 to axially translate within the enclosure, as will be described in more detail below. Base 44 has a coupling slot 46 formed in its lower edge.

The coupling arm 36 which carries the sample-collecting head 34 is attached at its proximal end to a shaft 64 having a square or other non-circular periphery. The shaft 64, in turn, is reciprocatably mounted in a rotating barrel 66 whose purpose will be described shortly. A coupling disk 60 is secured out or near the junction between the proximal end of the coupling arm 36 and the distal end of the shaft 64. As best seen in FIGS. 2A and 2B, the coupling disk 60 is received in the coupling slot 46 on the lower edge of the carriage base 44. In this way, as the user axially advances and/or retracts the carriage 42 using the thumb button 26, the base will push or pull the coupling disk 60 which in turn axially advances and/or retracts the sample-collecting head 34. The rotating barrel 66 will not move axially relative to the enclosure 12 but will be able to rotate the shaft 64 as the barrel itself is rotated by knob 30, as will be described in more detail below. In particular, shaft 64 will be “keyed” to the barrel so that the shaft and barrel will rotate together. While a square shaft received in a square receptacle within the barrel is illustrated, any conventional manner for rotationally keying the two elements could be used.

Referring now in particular to FIGS. 4 and 5, rotation of the rotating barrel 66 by the knob 30 is achieved through a gear chain comprising a drive gear 68 and follower gear 70. The knob 30 is directly connected to the drive gear 68 by a connector pin 78 (FIG. 5), and the drive gear 68 is mounted on a bearing pin 72 which is received in a supporting cylinder 74 which in turn is mounted between a pair of collector pins 78, as best seen in FIG. 3. Thus, the drive gear 68 will rotate at a 1:1 ratio as the knob 30 is rotated. Gear teeth 68a on the drive gear 68 directly engage gear teeth 70a on the follower gear 70, as best seen in FIG. 5. The drive gear 68 has a larger diameter than the follower gear 70, so the drive gear:follower gear rotation ratio is typically in a range from 1:4 to 1:12. As the rotating barrel 66 is fixed to the drive gear 70 so that they co-rotate, each of rotation of the knob 30 will cause the rotating barrel 66, the shaft 64, the coupling arm 36, and the sample-collecting head 34 to rotate from 4 to 12 times or more. Such increased rotation of the sample-collecting head is advantageous as it enhances the collection of cells and other biological specimens by the sample-collecting head.

The channel guides 48 of the carriage 42 are received on rails 58 formed on the inner surfaces of the half shells 40 of the enclosure 12. The cam element 52 on the bottom of the thumb button 26 will engage the ratchets 54 when the thumb button is in its unbiased configuration, i.e. is not depressed by the thumb, as shown in FIG. 5. Thus, when the thumb button 26 is not pressed downwardly, the carriage 42 will be held axially in place. By pressing down on the thumb button 26, however, the cam element 52 is released from the ratchets 54, allowing the user to advance and retract the carriage by sliding the thumb button axially along the slot 28.

Referring now to FIGS. 6A through 11B, the sample-collecting head of the present invention may take a variety of different forms. For example, as shown in FIGS. 6A and 6B, a sample-collecting head 100 may comprise a foldable backing 102 having a stem or other coupling arm 104 attached to a proximal surface opposite to a cell-collecting surface 106. A plurality of protrusions or fingers 108 may be formed over the cell-collecting surface 106, and the protrusions/fingers may be surrounded by a lip 110. The foldable backing 102 is typically circular or disk-shaped, although other shapes could also be used. The lip 110 will typically extend completely about the circular or other periphery, and the protrusions/fingers 108 will be formed in a three-dimensional pattern over the surface. The sample-collecting head 100 is shown in a sectional view for easier understanding.

The sample-collecting head 100 will be in its deployed or unfolded configuration, as shown in FIG. 6A when it is extended distally of the enclosure of the sample collector. Prior to and after use, however, the sample-collecting head 100 will be folded so that it can both be contained within the enclosure 12 and be drawn proximally back into the enclosure 12 through opening 32 after use. The folded configuration is particularly useful as the protrusion/fingers 108 which have engaged and contained much of the cell or other tissue sample, are contained within an interior of the folded surface which is protected by the surrounding lip 112.

Referring now to FIGS. 7A and 7B, an alternative sample-collecting head 120 is constructed similarly to that of FIGS. 6A and 6B except that protrusions 126, 128, and 132 have a variety of lengths, with the longer protrusions/fingers typically located near a center of a foldable backing 122 which is typically shaped as a disk. The sample-collecting head 120 typically also includes a lip structure 130 which surrounds a cell-collecting surface 126. A stem or rather a coupling arm 130 is attached to a rear surface of the foldable backing 122.

As described thus far, the foldable backings and the finger protrusions may be formed separately, typically from different materials. For example, the foldable backings 102 and 122 may be formed from pliable polymers which are structurally sufficient to withstand folding, unfolding, and passage through the opening 32 in the enclosure 12. The protrusions or fingers in contrast, may be formed from sponge-like or other softer materials which are configured to engage tissue and collect cells and other biological sample from the tissues in or surrounding the cervical os.

In contrast, as shown in FIGS. 8A and 8B, a sample-collecting head 140 may be formed as an integrated structure, typically from a sponge-like or other material suitable for directly engaging the cervical os and surrounding tissue. The geometry of the sample-collecting head 140, however, may be similar to those described earlier, for example having a single protrusion or finger 146 on a cell collecting surface 150 of a foldable backing 142 and a stem 144 opposite thereto. A surrounding lip 148 may also be provided. Folding of the sample-collecting head 140, as shown in FIG. 8B, will cause the surrounding lip 148 to fold inwardly to protect the single protrusion/finger 146, as with previous embodiments.

A further, alternative sample-collecting head 160 may comprise a plurality of protrusions/fingers 162 which are collected at their proximal ends and which diverge radially outwardly in a distal direction. A stem 146 may be attached to the region where the protrusions/fingers 162 are connected at their respective bases. The protrusions/fingers 162 may simply collapse or fold together when radially constrained, as shown in FIG. 9B.

In yet another embodiment, a sample-collecting head 170 as illustrated in FIGS. 10A and 10B. may be in the form of a sponge or similar absorptive material 172 having an ovoid shape with a stem 174 at its proximal end. While the ovoid sponge 172 will not fold as with previous embodiments, it may be radially constrained in configuration shown in FIG. 10B when it is maintained within the enclosure.

As a final example, a sample-collecting head 180 may comprise a sponge structure 182 having a conical cell-collection surface 184 at a distal end and a stem 186 attached at a proximal end, as shown in FIG. 11A. The sponge collection head 80 will not fold but will usually deform into a narrower configuration, as shown in FIG. 11B.

Referring now to FIGS. 12A through 12F, use of the sample collector 10 of the present invention for collecting a cell, tissue, or other sample from a user's cervical os CO will be described. The following steps are suitable for performance by a user to collect her own cell or other samples, typically without aid or assistance from any other person. The user may initially take the sample collector 10 in one hand, as shown in FIG. 12 A, and insert the distal region 18 into her vaginal canal VC through her vaginal opening VO, as shown in FIG. 12B. The distal region 18 will be inserted until the flange or other insertions stop 24 engages tissue surrounding the vaginal opening VO which prevents further insertion. The insertion depth will typically be within the ranges described above.

Once insertion as shown in FIG. 12B has been achieved, the user will advance the slider 24 in a distal direction, as shown by the arrow in FIG. 12C, to advance the sample-collecting head 34 until it engages her cervical os CO. The slider 24 may be fully or partially advanced, depending on when the sample-collecting head engages the tissue surrounding the cervical os CO. After the sample-collecting head 34 is properly positioned, the user will release the thumb button 26 causing the cam element 52 to lock with ratchets 54 as shown, for example, in FIG. 2B, to fix the sample-collecting head 34 in place relative to the enclosure 12.

After the sample-collecting head 34 has been locked in place, the user will then rotate knob 30 in order to cause the sample-collecting head 34 to rotate against the tissue surrounding the os CO, as shown by the arrow in FIG. 12D. Usually, the user will hold the enclosure in one hand while rotating the knob 30 in her other hand. In contrast, axial advancement of slider 24 will typically be achieved by the user with the hand which holds the enclosure 10, although two hand deployment may also be used.

After the user rotates the sample-collecting head a sufficient number of times, typically rotating the knob 30 from 1 to 10 times, which in turn causes a rotation of the sample-collecting head in the range from 5 rotations to 10 rotations (depending on the gear ratio, if any), the sample-collecting head may be axially retracted back within the enclosure 12 through the distal opening 32, as shown in FIG. 12E. The sample collector 10 may then be removed from the vaginal canal VC by the user, as shown in FIG. 12F. The sample collector 10 at the point holds the sample-collecting head 34 within its interior, and the sample-collecting head is ready for transfer to a suitable container for transport to a testing laboratory or other facility.

Referring now to FIGS. 13A through 13F, the sample-collecting head 34 may be transferred to a collection receptacle 190 by performing the following steps. First, the distal region 18 of the enclosure 12 may be introduced into an aperture 192 of the collection receptacle 190. As best shown in FIG. 13B, the distal region 18 may be inserted until its outer periphery engages and seals against an inner periphery of the aperture 192. Such sealing inhibits or prevents contamination of the sample-collecting head 34 as it is introduced into the container 190.

As shown in FIG. 13C, the sample-collecting head 34 is introduced into a preservative/culture solution 196 within an interior of the enclosure by advancing slider 24 in the manner similar to sample collection. The slider 24 is advanced until the collection head is fully immersed in the preservative solution 196.

The sample-collecting head 34 is then detached from the sample collector 10. Advantageously, detachment may be effected using a pair of blades 194 which may be externally closed by the user to sever the coupling arm or stem 36. Shown in FIG. 13D, the blade as manually squeezed inwardly (using external push tabs to avoid contamination) to shear the stem 36, leaving a partial stem portion 198 within the interior of the collection receptacle 190. The sample collector 10 may then be removed from the aperture 192, and the aperture may be closed by a cover 200 which covers the aperture as well as the exterior portions of the cutting blades 194. The cover 200 is then secured so that it remains in place until removal at a laboratory or other analytical facility. The covered receptacle, as shown in FIG. 13F, is suitable for transportation by normal delivery routes.

In an alternative embodiment, a sample collector 300 is provided with wheel-type axial and rotational actuators in place of the actuators described with reference to FIGS. 1A-5 above. The sample collector 300 includes an enclosure 304 (shown in broken line in FIG. 14) similar to enclosure 12 described previously. A sample-collecting head 310 is mounted on a distal end of a stem 306, where the stem can be advanced and retracted to selectively deploy and recover the sample-collecting head 310 in a manner similar to that described previously.

In place of the axial slider described previously, however, the sample collector 300 uses a toothed axial drive wheel 324 to engage and drive a flexible ratcheted section 312 of the stem 306. An upper portion of the toothed axial drive wheel 324 extends through a slot or other aperture in the enclosure so that a user can manually turn the drive wheel. By turning the toothed axial drive wheel 324 a counter-clockwise direction (typically using a thumb), the stem 306 and sample-collecting head 310 can be fully retracted, as shown in FIG. 15A. By manually turning the drive wheel 324 in a clockwise direction, in contrast, the stem 306 and sample-collecting head 310 can be fully advanced, as shown in FIGS. 14 and 15A. A proximal portion of the ratcheted section 312 of the stem 306 is typically coupled to a spool 320, as shown in FIG. 15A, so the ratcheted section can be rolled and unrolled as the stem is retraced and advanced.

The sample-collecting head 310 can be rotated by manually turning a toothed rotational drive wheel 330 about a longitudinal axis 332. The toothed rotational drive wheel 330 engages an axially splined region 314 of the stem 306. The rotational drive wheel 330 extends through an opening in the enclosure 304 in a manner similar to the toothed axial drive wheel 324. Typically, the axially splined region 314 will be rotationally coupled to the ratcheted region 312 of the stem so that the axially splined region can be rotated without rotating the ratcheted region. Alternatively, the ratcheted region could be ribbed (rather that having axially aligned ratchets) to accommodate rotation just as the splines accommodate axial advancement and retraction of the stem 306. An extension region 316 of the stem 306 extends through an opening 308 in the enclosure and carries the sample-collecting head in a manner similar to the previous embodiments.

In still other embodiments, the spool 320 could be configured to be rotated directly by the user. By doing so, the axial drive wheel 324 could be eliminated and an alternate drive wheel, knob, lever, or the like (not illustrated) coupled to the spool 320 for axial advancement and retraction of the stem 306.

In still other embodiments, a camera, an optical fiber, and/or other image sensor or system could be incorporated into the device to enable real time and/or archival visual or other imaging of the cervix and vaginal vault. The image sensor could provide optical imaging, near infrared imaging, contour imaging, ultrasound imaging, OCT imaging, or any other form of imaging. Such imaging could provide data which could be used either for (i) direct visualization to detect gross lesions/pathology or (ii) to help guide the collecting device to the cervical os (based on an automated feedback tracking software which could align the collecting device to the cervical os). The image sensor could be (i) integrated into the center of the flexible stem which encompasses the collecting device or (ii) on the tip of the enclose which gets inserted into the vaginal canal. Depending on the type of image sensor, a LED or other light source could also be provided. The imaging sensor could be coupled via a wired or wireless link back to a receiver which could be housed in the enclosure of the device or elsewhere, e.g. either through wires/optical cables or via WIFI/Bluetooth. The information from the receiver could be viewed, stored, analyzed on a local device, such as a computer, mobile phone, pad or other smart device, and/or could be sent to a centralized archive (e.g. on the cloud) for analysis and storage. With a local device, this information could also be used so the user can manually adjust the device in her vagina for correct alignment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for self-collection of a biological sample, said device comprising:
an enclosure configured to be gripped in a hand of a user and inserted into the user's vaginal canal, said enclosure having an opening at a distal end thereof;
a sample-collecting head configured to be advanced and retracted through the opening;
a translational actuator on a side of the enclosure, said translational actuator being coupled to the sample-collecting head and being configured to be engaged by the one or another of user's hands to advance the sample-collecting head through the opening to a location proximate the user's cervical os and to retract the sample-collecting head through the opening into an interior of the enclosure; and
a rotational actuator configured to be rotated by one or other of the user's hand to rotate the sample-collecting head while the enclosure is in the user's vaginal canal and is being held by one or other of the user's hands.

2. A device as in claim 1, wherein a distal region of the enclosure tapers a distal tip.

3. A device as in claim 2, wherein the distal region of the enclosure conforms to a vaginal anatomy.

4. A device as in claim 3, wherein the opening in the enclosure is at the distal tip.

5. A device as in claim 1, wherein the sample-collecting head comprises one or more of a brush, a sponge, a protrusions, bristles, flocculated bristles, and combinations thereof.

6. A device as in claim 1, wherein the sample-collecting head comprises a foldable backing having a forwardly facing sample-collecting surface, wherein the sample-collecting head is folded when in the interior of the enclosure and unfolds as it is advanced through the opening.

7. A device as in claim 6, wherein the sample-collecting head is configured to refold when retracted back into in the interior of the enclosure.

8. A device as in claim 1, further comprising a slidable carriage within the enclosure, wherein the slidable carriage is coupled to both the sample-collecting head and the translational actuator so that advancing and retracting the translational actuator advances and retracts the sample-collecting head.

9. A device as in claim 8, wherein the translational actuator comprises a slider having a thumb button and a coupling arm which attaches the thumb button to the slidable carriage, wherein the coupling arm is slidably received in an axial slot in a wall of the enclosure.

10. A device as in claim 9, wherein the axial slot has ratchets along an axial edge thereof and wherein the coupling arm is biased to engage the ratchets to immobilize the slider and the slidable carriage when the thumb button is released and to disengage the ratchets when the thumb button is depressed to advance and retract the slider and the slidable carriage.

11. A device as in claim 1, wherein the rotational actuator is coupled to the sample-collecting head by a rotatable barrel and a shaft, wherein the shaft is keyed to the rotatable barrel to allow axial extension and retraction of the sample-collecting head relative to the barrel while the rotatable barrel is rotated by the rotational actuator.

12. A device as in claim 1, wherein the rotational actuator is coupled to the rotatable barrel by a gear train which rotates the barrel from four to 12 times for each rotation of the rotational actuator.

13. A device as in claim 1, further comprising an insertion stop disposed on an exterior of the enclosure at a location spaced proximally of the distal end.

14. A device as in claim 1, wherein the rotational actuator comprises a knob disposed at a proximal end of the enclosure.

15. A system for collecting and transporting a cervical sample, said system comprising:
a device for self-collection of a cervical sample as in any one of claim 1;
a collection receptacle having an aperture configured receive the distal end of the enclosure to allow the sample-collecting head to be advanced through the opening of the enclosure and into an interior volume of the collection receptacle; and
a cover configured to be sealed over the aperture of the collection receptacle after the sample-collecting head has been detached from the device and released into the interior of the interior volume of the collection receptacle.

16. A system as in claim 15, wherein the aperture is configured to seal about an exterior of the enclosure of the device for self-collection.

17. A system as in claim 16, wherein the collection receptacles further comprising cutters on the receptacle configured to detach the sample-collecting head from the device for self-collection of a cervical sample while the aperture remains sealed about the exterior of the enclosure.

18. A method for self-collection of a cervical sample by a user, said method comprising:
gripping an enclosure;
inserting a distal end of the enclosure into the user's vaginal canal;
advancing a sample-collecting head from an interior of the enclosure through the opening to engage tissue proximate the user's cervical os;
rotating the sample-collecting head while the enclosure remains in the user's vaginal canal to collect sample from the tissue;
retracting the sample-collecting head back into the interior of the receptacle;
withdrawing the distal end of the enclosure from the user's vaginal canal after the sample-collecting head has been retracted back into the interior of the enclosure;
wherein all steps are performed by the user.

19. A method as in claim 18, wherein the user grips the enclosure in one hand and inserts the distal end of the enclosure into the vaginal canal using the one hand.

20. A method as in claim 19, wherein the user uses the one hand or another hand to advance a translational actuator on an exterior of the enclosure to advance the sample-collecting head from an interior of the enclosure through the opening to engage tissue proximate the user's cervical os.

21.-32. (canceled)
